(19) United States
(12) Patent Application Publication
(10) Pub. No.: US 2024/0390311 A1
(43) Pub. Date: Nov. 28, 2024

(54) COMPOSITIONS AND METHODS FOR AMELIORATING MEDICAL CONDITIONS RELATING TO CORONAVIRUS INFECTIONS

(71) Applicant: The Regents of the University of California, Oakland, CA (US)

(72) Inventors: Daniel L. KAUFMAN, Los Angeles, CA (US); Jide TIAN, Los Angeles, CA (US)

(73) Assignee: The Regents of the University of California, Oakland, CA (US)

(21) Appl. No.: 18/694,139

(22) PCT Filed: May 30, 2022
(86) PCT No.: PCT/US22/31485

§ 371 Date: Mar. 21, 2024

Related U.S. Application Data
(63) Continuation-in-part of application No. PCT/US21/51965, filed on Sep. 24, 2021.
(60) Provisional application No. 63/083,277, filed on Sep. 25, 2020.

(51) Int. Cl. A61K 31/197; A61K 45/06; A61P 31/14

(52) U.S. Cl. CPC A61K 31/197; A61K 45/06; A61P 31/14

(57) ABSTRACT

Use of GABA-receptor agonists, either alone or with one or more positive PAMs, anti-inflammatory compounds, and/or antiviral treatments, e.g., one that limits viral replication or impacts other viral functions, to ameliorate, treat, and/or prevent illness arising from infections, including bacterial, fungal, and/or viral infections, in patients, including to: ameliorate infection-related medical conditions; ameliorate and/or prevent coronavirus-related medical conditions; inhibit viral replication; inhibit corona virus replication; ameliorate and/or treat coronavirus-induced medical conditions; ameliorate and/or prevent respiratory virus-related medical conditions; and ameliorate and/or modulate dysregulated immune responses.

### CROSS-REFERENCE TO RELATED APPLICATION

The application is a Continuation-in-Part application of PCT/US2021/051965, filed on Sep. 24, 2021, which claims the benefit of U.S. Provisional Application Ser. No. 63/083,277, filed Sep. 25, 2020, which are hereby incorporated by reference herein in their entirety, including any figures.

### STATEMENT OF FEDERALLY-SPONSORED RESEARCH OR DEVELOPMENT

This invention was made with government support under Grant Number DE029020, awarded by the National Institutes of Health, and Grant Number W81XWH-20-1-0097, awarded by U.S. Army, Medical Research and Materiel Command. The government has certain rights in the invention.

### BACKGROUND OF THE INVENTION

Infections, be they bacterial, fungal, and/or viral, can result in harmful medical conditions. For example, severe acute respiratory syndrome coronavirus 2 (“SARS-CoV-2”) is a virus responsible for the coronavirus disease (“COVID-19”). As of Sep. 14, 2020, there have been over 200,000,000 confirmed cases of COVID-19 reported to the World Health Organization, and nearly 5,000,000 deaths. Despite the great success of vaccines to reduce serious illness due to SARS-CoV-2 infection, additional therapeutic approaches are urgently needed due to break-through infections, vaccine hesitancy, and new viral variants. New antiviral drugs hold great promise to help reduce severe disease and mortality due to COVID-19, however, these drugs may not become readily available in developing countries and they may be less effective against coronaviruses that emerge in the future. The identification of new therapeutics that have established safety records, are inexpensive, and do not have special storage requirements could be especially helpful for reducing COVID-19-associated morbidity and mortality worldwide.

There is an urgent need for compositions and methods that: ameliorate and/or prevent coronavirus-related medical conditions; inhibit viral replication; inhibit coronavirus replication; ameliorate and/or treat coronavirus-induced medical conditions; ameliorate and/or prevent respiratory virus-related medical conditions; and ameliorate and/or modulate dysregulated immune responses in patients suffering from an infection.

### SUMMARY OF THE INVENTION

The claimed invention uses gamma-aminobutyric acid (“GABA”)-receptor agonists to ameliorate, treat, and/or prevent illness arising from infections, including bacterial, fungal, and/or viral infections. The claimed invention includes methods that: ameliorate infection-related medical conditions; ameliorate and/or prevent coronavirus-related medical conditions; inhibit viral replication; inhibit coronavirus replication; ameliorate and/or treat coronavirus-induced medical conditions; ameliorate and/or prevent respiratory virus-related medical conditions; and ameliorate and/or modulate dysregulated immune responses in patients suffering from an infection by administering a GABA-receptor agonist, either alone or with one or more positive allosteric modulators (“PAMs”), anti-inflammatory compounds, and/or antiviral treatments, e.g., one that limits viral replication or impacts other viral functions.

### BRIEF DESCRIPTION OF THE DRAWINGS

FIG. 1A show daily changes in percent body weights post-infection (% of day 1). GABA treatment reduced body weight loss and death rate in MHV-1 infected mice. p<0.0001 for GABA0 and GABA3 vs. control. GABA0 vs. GABA3 p=0.175 by repeated measure ANOVA.

FIG. 1B show daily percent of surviving mice in each group (control and treatment groups). Data shown is from two separate studies with 4-5 mice/group; N=9 mice in control group, 10 mice in GABA-treated groups. GABA treatment increased survival following MHV-1 infection. p=0.002 and 0.001 for GABA0 and GABA3 vs. control, respectively by log rank test. GABA0 vs. GABA3 p=0.31.

FIG. 2 shows mean clinical scores+/−SEM of each group from two separate experiments in which mice were infected with MHV-1 and given plain water or water with GABA and monitored daily for the severity of their illness. GABA treatment reduced illness scores in MHV-1 infected mice. p<0.001 for GABA0 and GABA3 vs. control. GABA0 vs GABA3 p=0.042 using the non-parametric Kruskal-Wallis test.

FIG. 3 shows the mean lung coefficient index for each group from two separate studies in which mice were infected with MHV-1 and given plain water or water with GABA and then lungs were harvested and weighed when an animal became moribund or at 14 days post-infection. GABA treatment reduced the lung coefficient index in MHV-1 infected mice. ***p<0.001 and **p<0.01 for GABA0 and GABA3 (respectively) vs. control water treated group.

FIG. 4A shows daily changes in mean percent body weights (% of day 0, ±SEM) post-infection with MHV-1 for mice given plain water or a GABA agonist. ***p<0.001 vs. control, computed by a RM ANOVA model.

FIG. 4B shows daily scores for the severity of their illness post-infection with MHV-1 for mice given plain water or a GABA agonist. The data shown are the mean illness scores±SEM for each group. P values are indicated for each treatment vs. the control as calculated by the Kruskal-Wallis test. *p<0.05, ***p<0.001.

FIG. 4C shows daily percent of surviving mice post-infection with MHV-1 for mice given plain water or a GABA agonist. Indicated p values vs. the control were calculated by the log-rank test.

FIG. 4D shows lung coefficient indexes post-infection with MHV-1 for mice given plain water or a GABA agonist. The lungs were harvested and weighed when an animal became moribund or at 14 days post-infection. The data shown are the mean lung coefficient index±SEM for each group. *p<0.05, **p<0.01, ***p<0.001 vs. the control water treated group by Student's t-test.

FIG. 5 shows daily percent of surviving mice in each group (control and two GABA treatment groups) of K18-hACE2 mice infected with SARS-CoV2. GABA treatment increased survival following SARS-CoV2 infection. p=0.049 and p=0.174 for 0.2 and 20 mg/mL GABA vs. control, respectively by log-rank test.

FIG. 6A shows longitudinal mean illness score of K18-hACE2 mice infected with SARS-CoV2 with and without GABA treatment. Disease severity was scored and compared between groups by fitting mixed-effect linear regression models with group and time as fixed effects (to compare means), and with group, time and group by time interaction as fixed effects (to compare slopes).

FIG. 6B shows combined percent survival of K18-hACE2 mice infected with SARS-CoV2 with and without GABA treatment from two independent studies with 5 mice/group which followed the mice for 7 or 8 days post-infection (n=10 mice/group total). Survival curves were estimated using the Kaplan-Meier method and statistically analyzed by the log rank test.

FIG. 6C shows the lung coefficient index of K18-hACE2 mice infected with SARS-CoV2 with and without GABA treatment. Mice that reached an illness score of 5 or survived to the end of the observation period were euthanized. Their lungs were dissected and weighed to calculate the lung coefficient index (the ratio of lung weight to body weight). The data shown are the mean lung coefficient index±SEM/N=5 mice/group. The p-value was determined by Student's T test.

FIG. 7 shows viral titer in lung tissue of K18-hACE2 mice infected with SARS-CoV2 with and without GABA treatment. Three days post-infection the right lung was harvested from each mouse and homogenized. The viral titer in lung tissue (100 mg) was determined by TCID₅₀ assay using Vero E6 cells. Black dots show viral titers for individual mice (determined in quadruplicate). Data shown are the mean virus titer (Log₁₀ TCID₅₀/100 mg lung tissue)+SD in mice given plain water (control) or GABA. N=10 mice/group. The p-value was determined by Student's T test.

FIG. 8 shows the concentrations of various cytokines and chemokines in serum samples taken from uninfected B6 mice and K18-hACE2 mice infected with SARS-CoV2 that were untreated or treated with GABA. For the K18-hACE2 mice, three days post-infection with SARS-CoV2, serum samples were collected from both mice that were untreated (labeled “SC”) or that were GABA treated (labeled “G”). In addition, sera from age-matched health control B6 mice were studied (labeled “HC”). The non-normally distributed data are shown in boxplots with the borders of the box indicating 1st and 3rd quartile of each group (n=10), the bolded line indicating the median. The data were analyzed by Wilcoxon rank-sum tests. *p<0.05, ***p<0.001.

FIG. 9A shows longitudinal mean illness score of K18-hACE2 mice infected with SARS-CoV2 with and without GABA treatment starting at 2 days post infection. Disease severity was scored and compared between groups by fitting mixed-effect linear regression models with group and time as fixed effects (to compare means), and with group, time and group by time interaction as fixed effects (to compare slopes).

FIG. 9B shows combined percent survival of K18-hACE2 mice infected with SARS-CoV2 with and without GABA treatment starting at 2 days post infection. Survival curves were estimated using the Kaplan-Meier method and statistically analyzed by the log rank test.

FIG. 9C shows the lung coefficient index of K18-hACE2 mice infected with SARS-CoV2 with and without GABA treatment starting at 2 days post infection. Mice that reached an illness score of 5 or survived to the end of the observation period were euthanized. Their lungs were dissected and weighed to calculate the lung coefficient index (the ratio of lung weight to body weight). The data shown are the mean lung coefficient index±SEM/N=9 mice/group. The p-value was determined by Student's T test.

### DETAILED DESCRIPTION OF THE INVENTION

γ-aminobutyric acid type A receptors (GABA_{A}-Rs) are a family of ligand-gated chloride channels which play key roles in neurodevelopment and neurotransmission in the central nervous system (CNS). GABA_{A}-Rs are also expressed by cells of the human and murine immune systems. While GABA is well-known as a commonly used neurotransmitter in the central nervous system (“CNS”), it is becoming increasingly appreciated that many immune cells express GABA receptors (“GABA-Rs”). The biological roles of GABA-Rs on immune cells is not yet well understood, but there is a growing body of evidence that the activation of these receptors generally has immunoregulatory actions. In the innate immune system, antigen presenting cells (“APCs”) express GABA-A-type receptors (“GABA_{A}-Rs,” which form a chloride channel) and their activation reduces APC reactivity [1, 2]. Neutrophils express GABA-B-type receptors (“GABA_{B}-Rs,” which are G-protein coupled receptors), which modulate their function [3]. In the CNS, microglia express both GABA_{A}-Rs and GABA_{B}-Rs and their activation reduces microglia responsiveness to inflammatory stimuli [4]. Alveolar macrophages express GABA_{A}-Rs and application of a GABA_{A}-R-specific agonist decreases the expression of many pro-inflammatory molecules in cultures of LPS-stimulated lung macrophages [5]. In the adaptive immune system, it has been shown that GABA-R activation promotes effector T-cell cycle arrest without inducing apoptosis [6]. In vivo, administration of GABA, or the GABA_{A}-R-specific agonist homotaurine, inhibits autoreactive Th1 and Th17 cells while promoting CD4⁺ and CD8⁺ Treg responses [7-9]. Taking advantage of these properties, it has been demonstrated the that administration of GABA or homotaurine inhibits disease progression in mouse models of type 1 diabetes (“T1D”), multiple sclerosis, and rheumatoid arthritis, and limits inflammation in a mouse model of type 2 diabetes [1, 6, 8-10]. There are currently several ongoing clinical trials that are testing GABA treatment in individuals newly diagnosed with T1D (NCT02002130, NCT03635437, NCT03721991, NCT04375020).

Patients who develop severe COVID-19 appear to mount weaker or delayed innate immune responses to SARS-CoV-2, which leads to excessive adaptive immune responses later that do not taper off appropriately [11-13]. This can lead to “cytokine storms,” disseminated intravascular coagulation, multiple organ dysfunction syndrome (“MODS”), and death. Studies of anti-CD3-activated human PBMC have shown that GABA inhibits IL-6, CXCL10/IP-10, CCL4, CCL20, and MCP-3 production [14]. Longitudinal studies of COVID-19 patients reveal that high levels of serum IL-6 and Th1, Th17, and Th2-secreted proteins are associated with progression to severe illness [11, 15]. Many of these biomarkers of severe illness have been shown to be reduced by GABA-R agonists in the aforementioned in vitro studies of human PBMC and/or mouse models of autoimmune diseases.

Prior to Applicants' invention, there was no information on whether GABA treatment would modulate the outcome of viral infections. Moreover, an NIH drug screening program found that GABA and GABA agonists had no effect on SARS infection or replication.

Applicants' claimed invention uses GABA-receptor agonists to impact medical conditions in a positive way for the patient, thereby providing a surprising, new, and useful approach to limiting, e.g., excessive immune responses in COVID-19 patients.

In an embodiment, provided is a method for ameliorating an infection-related medical condition, comprising administering to a patient in need thereof an effective amount of a GABA-receptor agonist.

In another embodiment, provided is a method for ameliorating and/or preventing a coronavirus-related medical condition, comprising administering to a patient in need thereof an effective amount of a GABA-receptor agonist.

In yet another embodiment, provided is a method for inhibiting coronavirus replication in a patient, comprising administering to a patient in need thereof an effective amount of a GABA-receptor agonist.

In another embodiment, provided is a method for ameliorating and/or treating a coronavirus-induced medical condition, comprising administering to a patient in need thereof an effective amount of a GABA-receptor agonist.

In yet another embodiment, provided is a method for inhibit viral replication in a patient, comprising administering to a patient in need thereof an effective amount of a GABA-receptor agonist.

In yet another embodiment, provided is a method for ameliorating and/or preventing a respiratory virus-related medical condition, comprising administering to a patient in need thereof an effective amount of a GABA-receptor agonist.

In another embodiment, provided is a method for ameliorating and/or modulating dysregulated immune response in a patient suffering from an infection, comprising administering to said patient an effective amount of a GABA-receptor agonist.

Infections ameliorated by the claimed invention include bacterial, fungal, and viral infections.

Viral infections ameliorated by the claimed invention include those caused by coronaviruses, such as those caused by any strain of viruses such as human coronavirus OC43 (“HCoV-OC43”) (β-CoV), human coronavirus HKU1 (“HCoV-HKU1”) (β-CoV), human coronavirus 229E (“HCoV-229E”) (α-CoV), human coronavirus NL63 (“HCoV-NL63”) (α-CoV), Middle East respiratory syndrome-related coronavirus (“MERS-CoV”) (β-CoV), severe acute respiratory syndrome coronavirus (“SARS-CoV”) (β-CoV), and SARS-CoV-2 (β-CoV).

By GABA-receptor agonist is meant an agonist of GABA_{A}-receptors, GABA_{B}-receptors, and/or GABA_{A-rho} receptors (formerly known as GABA_{C}-receptors).

GABA_{A}-receptor agonists include: α₅IA, adipiplon, beta-alanine, bretazenil, CL-218,872, (−)-epigallocatechin-3-gallate, GABA, gaboxadol, homotaurine, imidazenil, isoguvacine, L-838,417, muscimol, piperidine-4-sulfonic acid, progabide, QH-ii-066, SL-651,498, taurine, zolpidem, and 3-acyl-4-quinolones.

GABA_{B}-receptor agonists include: baclofen, CGP-44532, GABA, gamma-hydroxybutyrate, isovaline, lesogaberan, phenibut, 3-aminopropylphosphinic acid, and 3-aminopropyl(methyl)phosphinic acid (SKF-97541).

GABA_{A-rho} receptor agonists include: CACA, CAMP, and GABOB.

Positive allosteric modulators (“PAMs”) include: alcohol (ethanol), barbiturates, benzodiazepines (such as alprazolam, diazepam, chlordiazepoxide), BHFF, BHF-177, BSPP, certain carbamates (such as carisoprodol, lorbamate, meprobamate), CGP-7930, cinacalcet, etomidate, fendiline, glutethimide, GS-39783, kavalactones, lanthanum, meprobamate, neuroactive steroids, neurosteroids, niacin/niacinamide, nonbenzodiazepines (such as eszopiclone, zolpidem), propofol, quinazolinones (such as diproqualone, etaqualone, methaqualone), riluzole, stiripentol, theanine, thienodiazepines, valerenic acid, volatile/inhaled anesthetics. GABA_{B}-receptor agonists are listed in italics.

Anti-inflammatory compounds include corticosteroids, such as dexamethasone.

The GABA-receptor agonist, PAM, and/or anti-inflammatory compound can be administered intradermally, intramuscularly, intraperitoneally, intravenously, orally, subcutaneously, sublingually, via aerosol delivery, or via a combination of delivery routes. Preferred routes include orally, sublingually, and/or via aerosol delivery. Administration of the GABA-receptor agonist and PAM and/or an anti-inflammatory compound can occur concurrently or in a staggered format.

In some embodiments, the GABA-receptor agonist is GABA administered in an amount of 1 ng/kg/day to 500 mg/kg/day. In particular examples, the GABA is administered in an amount of about 1 ng/kg/day-500 mg/kg/day, 10 ng/kg/day-500 mg/kg/day, 50 ng/kg/day-500 mg/kg/day, 100 ng/kg/day-500 mg/kg/day, 200 ng/kg/day-500 mg/kg/day, 400 ng/kg/day-250 mg/kg/day, 750 ng/kg/day-100 mg/kg/day, 1-1000 μg/kg/day 50-1500 μg/kg/day, 100-1000 μg/kg/day, 150-500 μg/kg/day, or 200-400 μg/kg/day.

Infection that is ameliorated by the claimed invention can result in one or more of: death, edema, excess immune response, fever, illness, increased secretion of inflammatory factors, increased viral replication, inflammation (such as inflammation of the lung, upper respiratory system, or central nervous system), lethargy, pneumonia, pneumonitis, and tussis.

Viral replication that is prevented and/or ameliorated by the claimed invention can result in one or more of: death, edema, excess immune response, fever, illness, increased secretion of inflammatory factors, increased viral replication, inflammation, lethargy, pneumonia, pneumonitis, and tussis.

Respiratory virus-related medical conditions that are prevented and/or ameliorated by the claimed invention can comprise one or more of: death, excess immune response, fever, illness, inflammation, lethargy, pneumonia, pneumonitis, and tussis.

Excessive immune responses that are prevented and/or ameliorated by the claimed invention can manifest as one or more of: increased alveolar fluid, inflammation of the lungs, and impaired lung function.

Applicants' invention is especially surprising because previous studies of GABA treatment were focused on autoimmune diseases. Autoimmune diseases progress slowly and the immune responses against the body's own tissue is very low. Moreover, during the development of T cells, T cells that strongly recognize self-proteins are eliminated in a process termed “central tolerance induction.” After this elimination, only T cells that very weakly recognize self-proteins are allowed to persist. T cells that do not recognize self-proteins are allowed to survive, and because there is no selection against them, T cells that strongly recognize foreign antigens persist and form the basis of our immunity against pathogens, such as coronaviruses. Therefore, when GABA is administered in autoimmune conditions it acts on low frequency T cells that weakly interact with self-proteins.

In contrast, after a viral infection, it is the innate immune system that first responds and adaptive immune responses do not arise until approximately a week later. The T cells that recognize foreign pathogens have “high affinity” to the antigens and these responses expand until they become a sizable fraction of the total T cell population. Prior to Applicants' invention there simply were no data on whether GABA treatment can assuage the strong innate and adaptive immune responses against a virus. Moreover, the activation of GABA-receptors on immune cells has only a weak effect on the immune cells—it is not like the immunodepletive therapies (anti-CD3) that lead to the death of T cells, or the anti-cytokine (anti-TNF) therapies that have robust effects on the immune cells and are in used in the clinic.

Autoimmune diseases studied prior to Applicants' invention are mediated by autoreactive T cells of the adaptive immune system. After a viral infection, however, it is the innate immune system that responds first, and adaptive immune responses arise approximately a week later. In mice, coronavirus infection causes illness almost the next day, and illness peaks about 5-7 days later, well before adaptive immune responses arise.

Because GABA has an immunosuppressive effect on autoimmune responses, those of skill in the art thought it was likely that GABA-receptor agonist treatment would suppress innate immune responses to the virus, allowing the virus to replicate to a greater extent and exacerbating disease, the opposite of what is desired. Moreover, in an effort to find new drugs to treat COVID-19, an NIH core screening facility screened thousands of compounds, including GABA and many GABA-receptor agonists and antagonists, for their ability to interfere with SARS-CoV-2 binding to its cellular receptor and to inhibit SARS-CoV-2 replication (https://opendata.ncats.nih.gov/covid19/databrowser). It was determined that GABA and other GABA-receptor agonists or antagonists did not interfere with SARS-CoV-2 interaction with its cellular receptor, nor its replication. These data argue against the hypothesis that GABA-receptor agonists may modulate coronavirus replication.

Thus, it was especially surprising that GABA-receptor agonists impact medical conditions in a beneficial way for the patient, thereby providing a new and useful approach to limiting excessive immune responses in COVID-19 patients.

### EXAMPLES

### Example 1-Modulation of Severity of Illness and Mortality Rate Via GABA Treatment

There is an urgent need for new treatments to prevent and ameliorate serious illness arising from excessive immune responses to SARS-CoV-2 in COVID-19 patients.

Many immune cells express GABA-Rs and their activation generally has immunoregulatory actions. In particular, treatment with GABA has been shown to inhibit Th1 and Th17 responses in mouse models of autoimmune disease, and to reduce human PBMC production of many of the inflammatory mediators that are associated with disease severity in COVID-19 patients. Because GABA-R agonists like GABA and homotaurine are safe for human consumption, stable, inexpensive, and available worldwide, they show promise as an effective treatment for COVID-19 patients.

This study evaluated a new therapeutic approach based on targeting gamma-aminobutyric acid (GABA) receptors (GABA-Rs). Specifically, this example studied whether oral GABA, a GABA-receptor agonist, treatment beginning at the time of murine hepatitis virus-1 (“MHV-1,” a pneumotropic coronavirus that has been widely used to model SARS-CoV infection in mice) inoculation, or starting three days post-inoculation (by which time signs of illness are apparent), could modulate the seriousness of the ensuing illness and the rate of mortality. This example shows that oral GABA treatment greatly reduced illness, lung inflammation, and death when administered at the time of MHV-1 inoculation or after the appearance of illness.

### Methods

Mice. Female A/J mice (7 weeks in age) were purchased from The Jackson Laboratory and maintained in microisolator cages and fed a standard diet and water ad libitum. One week after arrival they were inoculated with MHV-1. The mice were immediately randomized and treated (or not treated) with GABA, as described below. This study was carried out in accordance with the recommendations of the Guide for the Care and Use of Laboratory Animals of the National Institutes of Health. The protocols for all experiments using vertebrate animals were approved by the Animal Research Committee at UCLA.

Reagents: GABA was purchased from Millipore-Sigma (stock #A2129, St. Louis, MO, USA).

Virus: Like SARS-CoV and SARS-CoV-2, MHV-1 is a pneumotropic beta-coronavirus of the group 2 lineage and is widely used as a safe model of SARS-CoV infection [16-19]. MHV-1 was used because unlike the MHV-JHM and MHV-A59 strains, which primarily infect the brain or liver, MHV-1 is pneumotropic. MHV-1 infection creates a lethal pneumonitis, similar to SARS-CoV-induced disease, in A/J mice. Intranasal inoculation with 5000 plaque-forming units (“PFU”) of MHV-1 in A/J mice induces an acute respiratory distress syndrome with a lethality rate of about 50%. The infected mice develop pathological features of SARS-CoV-2, including high levels of pulmonary cytokines/chemokines, pneumonitis, dense macrophage infiltrates, hyaline membranes, fibrin deposits, accompanied by loss of body weights and respiratory distress [16-19]. MHV-1, DBT cells, and HeLa-CECAM1 cells to grow and titer the virus were generously provided by Dr. Stanley Pearlman. MHV-I was prepared and titered as previously described [16-19].

Viral infection and GABA treatment: At 8 weeks in age, female A/J mice were anesthetized and inoculated intranasally with 5000 PFU MHV-1 in 50 μl cold Dulbecco's modified Eagle's medium (“DMEM”). The mice were immediately randomized and provided with plain water (controls) or water that contained GABA (20 mg/ml) for the entirety of the observation period. Another group of MHV-1 inoculated mice received plain water for three days, by which time they displayed signs of illness, and then were placed on GABA-containing water for the rest of the observation period. Body weights were monitored daily beginning on the infection day and up to 14 days post-infection.

Illness scoring. Individual mice were monitored for illness development and progression, which were scored on the following scale: 0) no symptoms, 1) slightly ruffled fur and altered hind limb posture; 2 ruffled fur and mildly labored breathing; 3) ruffled fur, inactive, moderately labored breathing; 4) ruffled fur, obviously labored breathing and lethargy; 5) moribund and death. The percent survival of each group of mice was determined longitudinally for each group. Mice with a disease score of 5 were weighed, euthanized, and their lungs removed and weighed for calculation of lung coefficient index (the ratio of lung weight to total body weight, which reflects the extent of edema and inflammation in the lungs). On day 14 post-infection, the surviving animals were weighed, euthanized, and their lungs were removed and weighed for determination of the lung coefficient index.

### Results

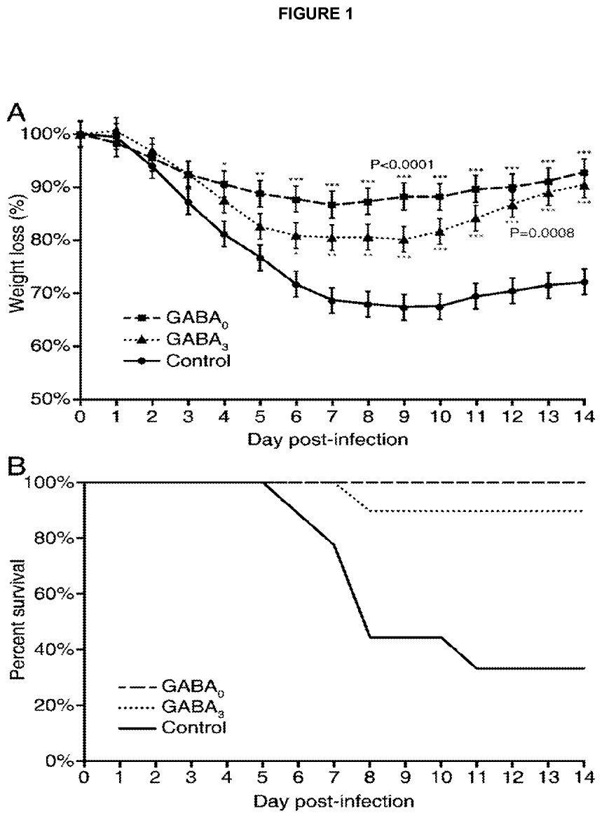

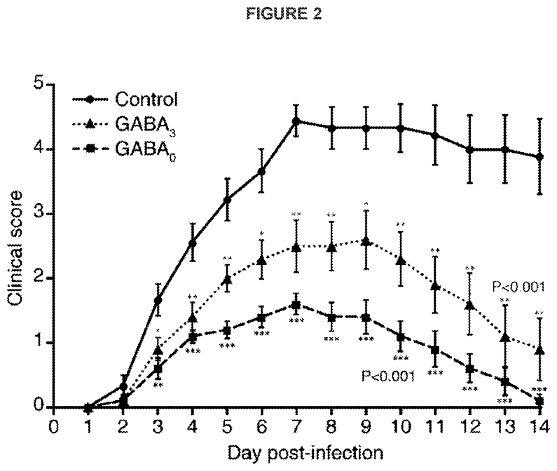

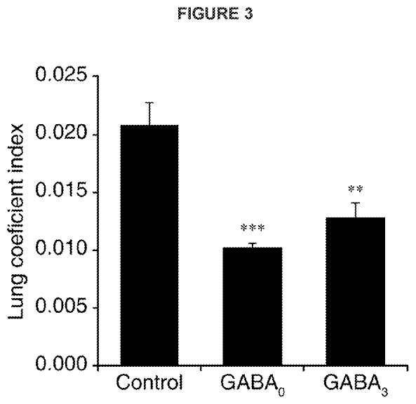

Following MHV-1 inoculation, mice receiving plain water began to progressively lose body weight each day. By day 6, the control group had lost an average of 23% of their weight, as expected [16-19]. At this time point, the mice that had been given GABA immediately after MHV-1 infection had lost an average of 11% of their body weight, and those given GABA three days after infection had lost an average of 17% of their body weight (FIG. 1A). After day 6, the mice in the control group began to succumb to their illness and only 3/9 mice survived on day 14 post-infection (FIG. 1B). In contrast to control mice, none of the mice given GABA starting immediately after MHV-1 inoculation died (FIG. 1B), and their body weight was on average only 7% below their starting weights 14 days post-infection (FIG. 1A). Of the mice given GABA 3 days post-infection, 1/9 mice died (on day 9), and their body weights at 14 days post-infection was 90% of their starting weight. The survival curves for each group are shown in FIG. 1B.

In terms of illness, MHV-1 infected control mice began to display signs of illness two days post-infection and rapidly became severely ill thereafter, with their illness peaking around day 7 post-infection. While most control mice died between days 6-11 post-infection, those that survived displayed only partial recovery from illness. In contrast, the mice receiving GABA immediately after inoculation developed only mild illness, with a highest average illness score of 1.6 on day 7 post-infection. Notably, illness in the mice given GABA at 3 days post-infection was also significantly reduced compared to that in the control group, and their maximum mean illness score was 2.5. Thus, GABA treatment immediately or 3 days after MHV-1 infection when the clinical signs of the disease appear, reduced the severity of coronavirus-induced illness and death.

The lung coefficient index reflects the edema and inflammation of the lung. The lung coefficient index of mice that were given GABA immediately after MHV-1 infection was 49% of that of control mice (p<0.001). The mice receiving GABA treatment beginning 3 days post-infection had a lung coefficient index that was 62% of that in the control mice (p<0.01). This provides an independent measure indicating that GABA treatment limited the MHV-1 induced pulmonary edema and inflammation in A/J mice.

### DISCUSSION

Together, the reduction in body weight loss, illness scores, death rate, and lung coefficient index indicate that GABA treatment can reduce illness severity and death rate following coronavirus infection, even when the treatment is initiated after symptoms appear.

Given that weaker and delayed interferon responses to the virus are associated with severe illness in COVID-19 patients [11] and GABA has anti-inflammatory effects, prior to Applicants' invention one would have anticipated that treatment with GABA immediately after MHV-1 infection might be deleterious by limiting or delaying innate immune responses. Applicants' invention is surprising in that early GABA treatment immediately after MHV-1 infection was very effective in preventing illness progression and death, suggesting a rapid effect of GABA on innate immune responses or the lung airway cells. The lung epithelial cells of mice and humans also express GABA_{A}-Rs [20, 21]. Activation of these GABA_{A}-Rs may lead to Cl-efflux, which would act to limit Ca²⁺ influx in these epithelial cells. Because many viruses, including coronaviruses, elevate intracellular Ca²⁺ concentrations in order to enhance viral replication [22, 23], the activation of GABA_{A}-Rs can limit MHV-1 replication.

Additionally, it has been shown that treatment with GABA, a GABA_{B}-agonist, or GABA_{A}-R positive allosteric modulators can reduce inflammation and improve alveolar fluid clearance and lung functional recovery in rodent models of acute lung injury [24-28].

Added benefits to Applicants' claimed invention include that GABA treatment was tested in hundreds of epilepsy patients for its ability to reduce seizures [29-31]. While it had no clinical benefit (probably because it cannot cross the blood brain barrier), it had no adverse effects in these long-term studies. A more recent phase Ib GABA oral dosing study also indicated that GABA is safe [32] and there are currently several ongoing clinical trials which are administering oral GABA to individuals with Type 1 Diabetes (“T1D”) (ClinicalTrials.gov Identifiers: NCT02002130, NCT03635437, NCT03721991, NCT04375020). In addition, the GABA_{A}-R specific agonist homotaurine was tested in a large long-term phase III clinical trial for Alzheimer's disease and while it was not effective it had an excellent safety record (see [8, 9] for a discussion of homotaurine's safety). Both GABA and homotaurine are inexpensive, stable at room temperature, and available world-wide making them excellent candidates for clinical testing as adjunctive treatments for, inter alia, COVID-19.

### Example 2-Effect of GABA_{A}-R Vs. GABA_{B}-R Agonists in MHV-1 Infected Mice

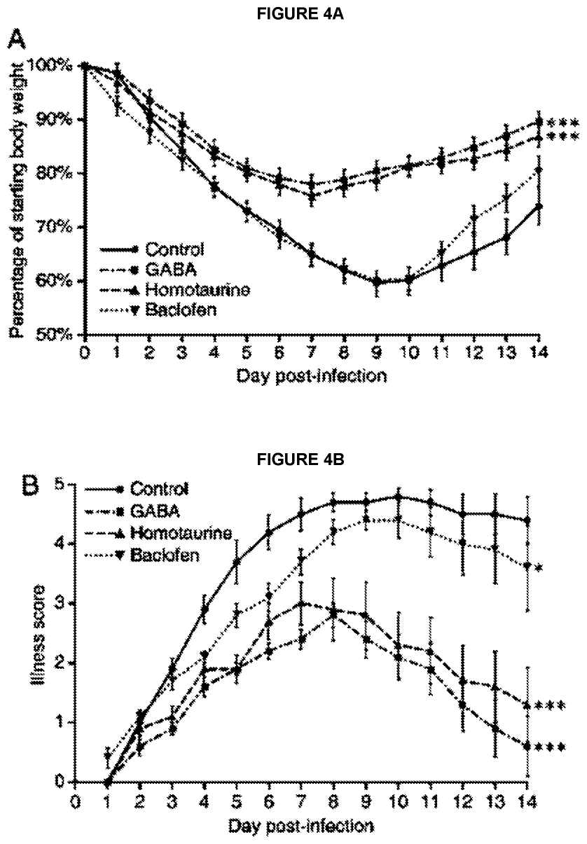

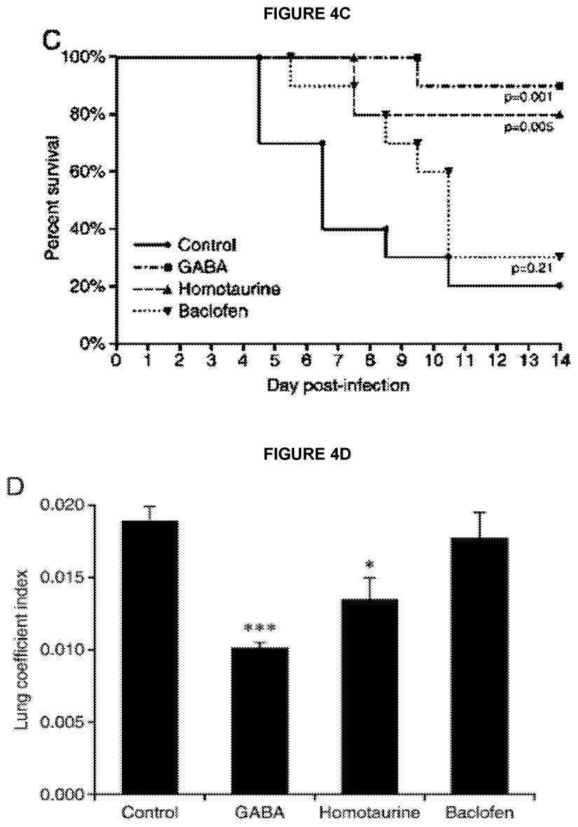

We assessed whether GABA's therapeutic effects were mediated through GABA_{A}-Rs, GABA_{B}-Rs, or both GABA-R subtypes. A/J mice were inoculated MHV-1 and given plain water or water containing GABA (2 mg/mL), a clinically applicable GABA_{A}-R-specific agonist (homotaurine, 0.25 mg/mL), or a GABA_{B}-R-specific agonist (baclofen, 0.25 mg/mL). We found that treatment with GABA or homotaurine significantly reduced the body weight loss (FIG. 4A), disease scores (FIG. 4B), death rate (FIG. 4C), and lung coefficient index in mice (FIG. 4D). Baclofen displayed a slight but significant ability to reduce illness scores; however, it did not significantly decrease the body weight loss, death rate and lung coefficient index in these mice relative to that of untreated controls. Thus, GABA's therapeutic effects are primarily mediated through GABA_{A}-Rs.

Looking at FIG. 4, panel A shows daily changes in mean %±SEM of body weights post-infection (% of day 0), ***p<0.001 vs. control, computed by a RM ANOVA model. Panel B shows daily scores for the severity of their illness. The data shown are the mean illness scores±SEM for each group. P values are indicated for each treatment vs. the control as calculated by the Kruskal-Wallis test. *p<0.05, ***p<0.001. Panel C shows daily percent of surviving mice in each group. Indicated p values vs. the control were calculated by the log-rank test. Panel D shows lung coefficient indexes. The lungs were harvested and weighed when an animal became moribund or at 14 days post-infection. The data shown are the mean lung coefficient index±SEM for each group. *p<0.05, **p<0.01, ***p<0.001 vs. the control water treated group by Student's t-test. For all studies, n=10 mice/group from two separate experiments.

### Example 3-Administration of a GABA-R Agonist Reduces the Severity of Pneumonia and Death Rates in SARS-CoV-2-Infected K18-hACE2 Transgenic Mice

### Trial 1:

We performed a study in which K18-hACE2 mice (N=5 mice/group) were inoculated with SARS-CoV-2 (1×10³ PFU) and placed on plain water or water with 0.2 or 20 mg/ml GABA. The mice were monitored for their disease progression and survival up to 7 days post-inoculation. The surviving mice were euthanized at day 7 post-infection.

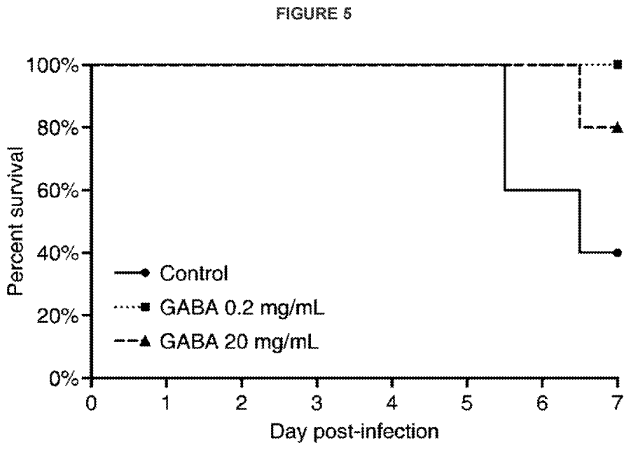

GABA treatment reduced the death rates in SARS-CoV-2 infected mice compared to that of mice given plain water (p=0.049 and p=0.174 for 0.2 and 20 mg/mL GABA vs. control, respectively by log-rank test). While 3/5 of the control mice died during the 7-day observation period, none of the mice treated with GABA 0.2 mg/mL died (p<0.05), and 1/5 of those given GABA 20 mg/ml died (FIG. 5).

Treatment with 0.2 mg/ml GABA also reduced the lung coefficient index (the ratio of lung weight to total body weight, which reflects the extent of edema and inflammation in the lungs) (p<0.01, data not shown). We expect that RT-qPCR analysis would demonstrate that viral N1 and N2 transcripts are lower in the lungs from GABA-treated vs. control mice harvested on days 6-7.

Collectively, these findings demonstrate that administration of a GABA-R agonist, such as GABA, can reduce death rates and pneumonia severity and improve outcomes in SARS-CoV-2 infected K18-hACE2 mice.

### Trial 2:

These studies were carried out in accordance with the recommendations of the Guide for the Care and Use of Laboratory Animals of the National Institutes of Health. The protocols for all experiments using vertebrate animals were approved by the Animal Research Committee at UCLA (Protocol ID: ARC #2020-122; Date Aug. 25, 2020-Aug. 24, 2023) and were carried out in compliance with the ARRIVE guidelines.

SARS-CoV-2 (USA-WA1/2020) was obtained from the Biodefense and Emerging Infections Resources of the National Institute of Allergy and Infectious Diseases. All in vivo studies of SARS-CoV-2 infection were conducted within a Biosafety Level 3 facility at UCLA or USC. SARS-CoV-2 stocks were generated by infection of Vero-E6 cells (American Type Culture Collection (ATCC CRL1586)) cultured in DMEM growth media containing 10% fetal bovine serum, 2 mM L-glutamine, penicillin (100 units/ml), streptomycin (100 units/ml), and 10 mM HEPES. Cells were incubated at 37° C. with 5% CO₂ and virus titer was determined as described below for tittering virus in lung homogenates.

Two independent test studies were performed, one at UCLA and a second at USC with 5 mice per treatment group in each study. Female K18-hACE2 mice (8 weeks in age) were purchased from the Jackson Laboratory. One week after arrival, mice were inoculated intranasally with SARS-Cov-2 (2,000 PFU or 2,000 TCID₅₀ (at USC and UCLA, respectively) in 20 μl Dulbecco's modified Eagle's medium. The mice were randomized into three groups of 5 mice and immediately placed in cages with water bottles containing 0 mg/mL (control), 0.2 mg/mL, or 2.0 mg/mL GABA (Millipore-Sigma, St. Louis, MO, USA; stock #A2129). The mice were maintained on those treatments for the remainder of each study. There was no statistically significant differences in longitudinal body weights or the amounts of water consumed among the different groups of mice.

The animals' behavior was monitored and severity of the disease was scored twice daily after infection. Illness development and disease progression were scored on the following scale: 0) no symptoms, 1) slightly ruffled fur and altered hind limb posture; 2) ruffled fur and mildly labored breathing; 3) ruffled fur, inactive, moderately labored breathing; 4) ruffled fur, inactive, obviously labored breathing, hunched posture; 5) moribund or dead. The percent survival of each group of mice was determined longitudinally. Mice with a disease score of 5 were weighed, euthanized, and their lungs removed and weighed for calculation of lung coefficient index (the ratio of lung weight to total body weight, which reflects the extent of edema and inflammation in the lungs). On day 7 or 8 post-infection, the surviving animals were weighed, euthanized, and their lungs were removed and weighed for determination of the lung coefficient index.

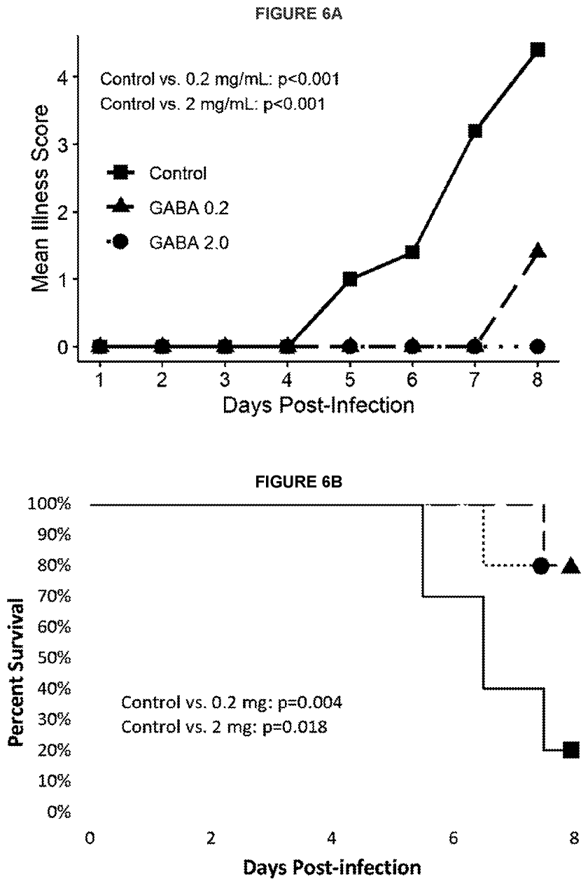

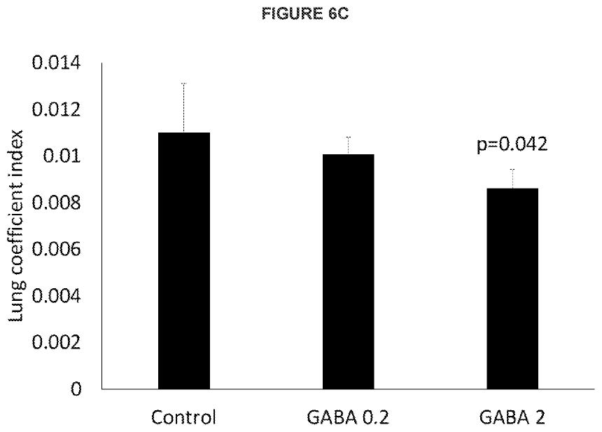

The SARS-Cov-2 infected control mice that received plain water developed overt signs of illness beginning around day 5 post-infection which increased in severity and often necessitated humane euthanasia prior to the end of the study (FIGS. 6A and 6B), similar to previous observations in this model. In contrast, mice receiving GABA displayed significantly reduced illness scores compared to the control mice (FIG. 6A). Only 20% of control mice survived until the pre-determined end of the studies 7-8 days post-infection. In contrast, 80% of the mice that received GABA at 0.2 or 2 mg/mL were surviving on days 7-8 post-infection (FIG. 6B, p=0.004 and p=0.018, respectively vs. the control).

At the time of euthanasia, each animal's lungs were excised and weighed. The ratio of lung weight to total body weight (i.e., the lung coefficient index) reflects the extent of edema and inflammation in the lung. We observed a significant reduction in the lung coefficient index scores in the group treated with 2.0 mg/mL GABA (FIG. 6C), indicating reduced (lung damage or inflammation).

Together, these studies demonstrate that GABA treatment can reduce disease severity, inflammation, and death in SARS-CoV-2 infected mice.

### Example 4-Administering GABA_{A}-R Agonist 2 Days Post-Infection

The study of Example 3 was repeated in a modified procedure aimed at evaluating the efficacy of GABA_{A}-R agonist treatment (such as GABA at 0.2 and 2.0 mg/mL doses) when the treatment is initiated 2 days post-infection (PI) as to opposed to at the time of infection. K18-hACE2 mice were infected with SARS-CoV-2 as described in Example 3 and two days later (near the peak of viral load in the lungs) mice received GABA through their drinking water (2 mg/mL), or were maintained on plain water for the remainder of the study. Compared with that of GABA-untreated control mice, GABA-treated mice displayed reduced longitudinal mean illness scores (overall p=0.002, FIG. 9A). At the end of the study on day 7, only 22% of control mice survived, while 88% of GABA treated mice had survived (p=0.004, FIG. 9B). GABA-treated mice also had a reduced lung coefficient index (p<0.001, FIG. 9C). This study demonstrates that initiating GABA treatment 2-days post-infection, near the peak of vial loads in the lungs, also decreases disease severity and death rates during the observation period.

### Example 5-Effect of GABA_{A}-R Agonist Treatment on Viral Load in the Lungs of SARS-CoV-2 Infected Mice at Time Points of Interest

This study was carried out in accordance with the recommendations of the Guide for the Care and Use of Laboratory Animals of the National Institutes of Health. The protocols for all experiments using vertebrate animals were approved by the Animal Research Committee at UCLA (Protocol ID: ARC #2020-122; Date Aug. 25, 2020-) Aug. 24, 2023) and were carried out in compliance with the ARRIVE guidelines.

SARS-CoV-2 (USA-WA1/2020) was obtained from the Biodefense and Emerging Infections Resources of the National Institute of Allergy and Infectious Diseases. All in vivo studies of SARS-CoV-2 infection were conducted within a Biosafety Level 3 facility at UCLA or USC. SARS-CoV-2 stocks were generated by infection of Vero-E6 cells (American Type Culture Collection (ATCC CRL1586)) cultured in DMEM growth media containing 10% fetal bovine serum, 2 mM L-glutamine, penicillin (100 units/ml), streptomycin (100 units/ml), and 10 mM HEPES. Cells were incubated at 37° C. with 5% CO₂ and virus titer was determined as described below for tittering virus in lung homogenates.

Female K18-ACE2 mice (9 weeks in age) were intranasally inoculated with 2,000 TCID₅₀ SARS-Cov-2 and placed on plain water or water containing GABA (2 mg/mL) for the rest of the study (N=10 mice/group). Three days post-infection, the mice were euthanized and individual blood samples were collected for preparing serum samples. A portion of their lower right lung was weighed and about 100 mg of wet lung tissue from each mouse were homogenized into 1 mL of ice-cold DMEM with 10% fetal calf serum with 1 mm glass beads using a Minilys homogenizer at 50 Hz for 1 min, followed by centrifugation. Supernatants were collected for virus titering. Vero E6 cells (1×10⁴ cells/well) were cultured in DMEM medium supplemented with 10% FBS in 96-well plates overnight to reach 80% of confluency and infected in quintuplicate with a series of diluted mouse lung homogenates in 100 μl of FBS-free DMEM medium at 37° C. for four days. The percentages of viral cytopathic effect areas were determined. The SARS-CoV-2 titers were calculated by the Reed and Muench method.

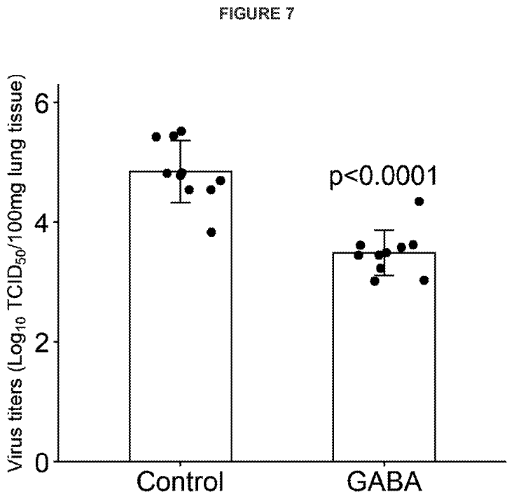

SARS-CoV-2 titers in the lungs of GABA-treated mice were on average a 1.36 log 10 (23-fold) lower than that in the lungs of mice that received plain water (p<0.0001, FIG. 7). Black dots show viral titers for individual mice (determined in quadruplicate). Data shown are the mean virus titer (Log₁₀ TCID₅₀/100 mg lung tissue)±SD in mice given plain water (control) or GABA. The p-value was determined by Student's T test. This study demonstrated that GABA treatment reduced viral loads in the lungs of SARS-CoV-2 infected mice.

### Example 6-GABA Treatment Modulates Early Cytokine and Chemokine Responses to SARS-CoV-2 Infection

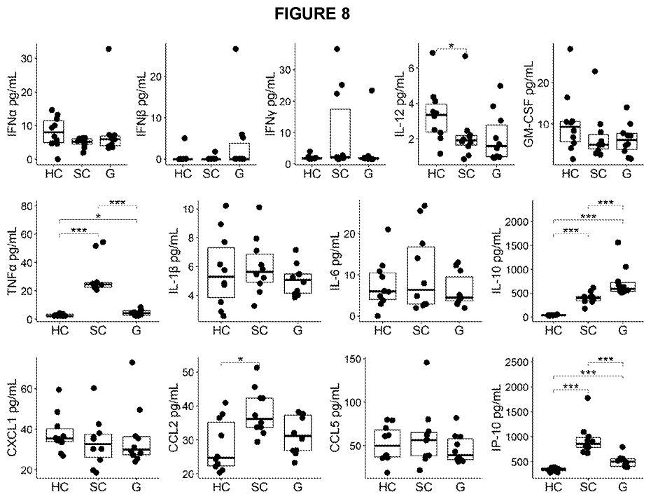

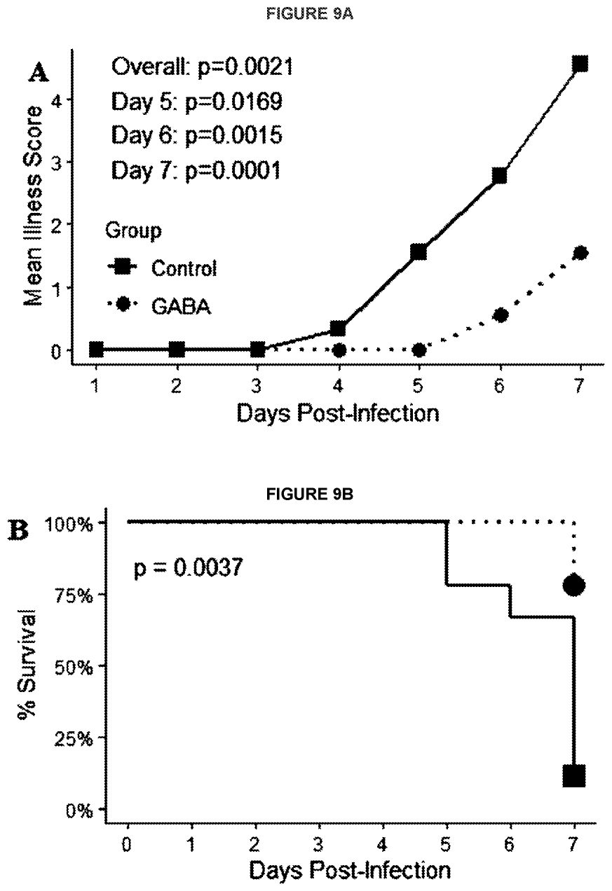

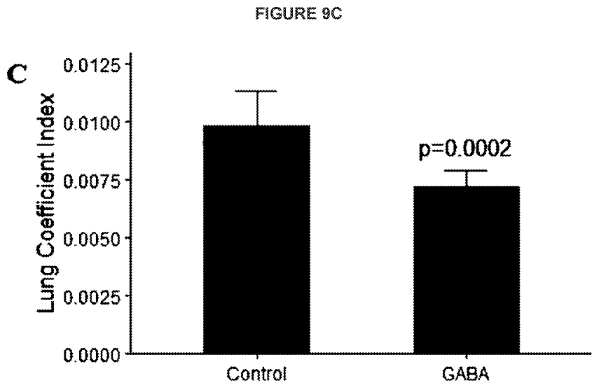

The effect of GABA treatment on serum cytokine and chemokine levels during SARS-CoV2 infection was assessed. Sera samples collected in Example 5 (collected three days post-infection from K18-hACE2 mice treated with 2 mg/mL GABA or untreated) were prepared and stored at −80° C. In addition, sera from age-matched health control (HC) B6 mice were studied. The levels of various serum cytokines and chemokines were determined by a bead-based multiplex assay using the LEGENDplex mouse anti-virus response panel (13-plex) kit (#740622, Biolegend, San Diego, USA), according to the manufacturer's instructions. Briefly, the control and experimental groups of serum samples were diluted at 1:2 and tested in duplicate simultaneously. After being washed, the fluorescent signals in each well were analyzed by flow cytometry in an ATTUNE NXT flow cytometer (Thermofisher). The data were analyzed using the LEGENDplex™ Data Analysis Software Suite (Biolegend) and the levels of each cytokine or chemokine in serum samples were calculated, according to the standard curves established using the standards provided. Resulting boxplots are shown in FIG. 8, with the “HC” group representing the B6 healthy control mice, the “SC” group representing the SARS-CoV2 infected K18-hACE2 that were untreated, and the “G” group representing the SARS-CoV2 infected K18-hACE2 that were treated with 2 mg/mL GABA.

The levels of serum IFNα, IFNβ, IFNγ, IL-12, or GM-CSF were not found to have a statistically significant difference between SARS-CoV-2 infected mice and that did or did not receive GABA treatment (FIG. 8). There was, however, some suggestion that GABA treatment elevated type I interferons in some mice because only 1/10 mice in the healthy control (HC) and in the SARS-CoV-2-infected control (SC) group had a detectable level of IFNβ, while 3/10 of the infected mice which received GABA (G) had detectable IFNβ levels and these were greater in magnitude than that found in the other groups (FIG. 8). The median and the mean levels of IFNα were also slightly elevated in the GABA treated vs. untreated SARS-CoV-2 infected group (p=0.14, FIG. 8). Conversely, 3/10 mice in the SC group displayed high levels of IFNγ vs 1/10 mice in the G group and the median and mean IFNγ levels were reduced in the GABA-treated vs. untreated SARS-CoV-2 infected mice (FIG. 8, p=0.25)

Analysis of pro-inflammatory and anti-inflammatory cytokines revealed that SARS-CoV-2 infection significantly increased the levels of serum TNFα in the SC group (p<0.001, FIG. 8). GABA treatment significantly mitigated TNFα production following infection (p<0.001, FIG. 8). There was no statistically significant difference in the levels of serum IL-1β and IL-6, although the levels of serum IL-1β and IL-6 in the GABA-treated group were slightly reduced compared with the SC group (FIG. 8). SARS-CoV-2 infection significantly increased the levels of serum IL-10 (FIG. 8). GABA treatment further significantly elevated the serum IL-10 levels above that in untreated SARS-CoV-2 infected mice (p<0.001, FIG. 8).

Analysis of serum chemokines revealed that untreated SARS-CoV-2 infected mice, but not the GABA-treated SARS-CoV-2 infected mice, displayed significantly higher levels of CCL2 relative to healthy controls (p<0.05), with the levels of CCL2 tending to be lower in GABA-treated vs. untreated-infected mice (p=0.07, FIG. 8). Moreover, GABA treatment led to significantly reduced levels of IP-10 (CXCL10) compared to that in untreated SARS-CoV-2 infected mice (p<0.001, FIG. 8). No statistically significant difference in the levels of serum CXCL1 and CCL5 was observed among these groups of mice.

Thus, GABA treatment shifted systemic cytokine and chemokine responses towards those associated with less risk for developing severe COVID-19 by increasing early type I responses in some mice, significantly reducing the levels of TNFα and IP-10, tending to reduce CCL2 levels, but enhancing IL-10 production in SARS-CoV-2 infected mice.

### Example 7-GABA-Receptor Agonist Treatment at the First Signs of Illness Improves Outcome

An effective amount of one or more GABA-receptor agonists, either alone or in combination with: one or more PAMs, anti-inflammatory compounds, and/or antiviral treatments, e.g., one that limits viral replication or impacts other viral functions, administered concurrently or in a staggered format to a patient in need thereof at the first signs of illness, improves outcome for the patient.

### Example 8-GABA-Receptor Agonist Treatment after Serious Illness Develops Improves Outcome

An effective amount of one or more GABA-receptor agonists, either alone or in combination with: one or more PAMs, anti-inflammatory compounds, and/or antiviral treatments, e.g., one that limits viral replication or impacts other viral functions, administered concurrently or in a staggered format to a patient in need thereof after serious illness develops, improves outcome for the patient.

### Example 9-GABA-Receptor Agonist Treatment Optimized for the Stage of Disease Process Improves Outcome

An effective amount of one or more GABA-receptor agonists, either alone or in combination with: one or more PAMs, anti-inflammatory compounds, and/or antiviral treatments, e.g., one that limits viral replication or impacts other viral functions, administered concurrently or in a staggered format to a patient in need thereof at a time optimized for the stage of disease process, improves outcome for the patient.

### Example 10-GABA-Receptor Agonist Treatment Optimized for the Stage of Disease Process Improves Outcome

An effective amount of one or more GABA-receptor agonists, either alone or in combination with: one or more PAMs, anti-inflammatory compounds, and/or antiviral treatments, e.g., one that limits viral replication or impacts other viral functions, administered concurrently or in a staggered format to a patient in need thereof at a time optimized for the stage of disease process, improves outcome for the patient.

### Example 11-GABA-Receptor Agonist Treatment Administered as Precision Medication Based Upon the Patient's Genotype and/or Biomarkers Improves Outcome

An effective amount of one or more GABA-receptor agonists, either alone or in combination with: one or more PAMs, anti-inflammatory compounds, and/or antiviral treatments, e.g., one that limits viral replication or impacts other viral functions, administered concurrently or in a staggered format to a patient in need thereof as precision medication based upon the patient's genotype and/or biomarkers, improves outcome for the patient.

### REFERENCES

1. Tian J, Yong J, Dang H, Kaufman D L. Oral GABA treatment downregulates inflammatory responses in a mouse model of rheumatoid arthritis. Autoimmunity. 2011; 44:465-70. PubMed PMID: 21604972.
2. Bhat R, Axtell R, Mitra A, Miranda M, Lock C, Tsien R W, Steinman L. Inhibitory role for GABA in autoimmune inflammation. Proc Natl Acad Sci USA. 2010; 107(6):2580-5. PubMed PMID: 20133656; PMCID: PMC2823917.
3. Rane M J, Gozal D, Butt W, Gozal E, Pierce W M, Jr., Guo S Z, Wu R, Goldbart A D, Thongboonkerd V, Mcleish K R, Klein J B. Gamma-amino butyric acid type B receptors stimulate neutrophil chemotaxis during ischemia-reperfusion. J Immunol. 2005; 174(11):7242-9. PubMed PMID: 15905570.
4. Lee M, Schwab C, McGeer P L. Astrocytes are GABAergic cells that modulate microglial activity. Glia. 2011; 59 (1):152-65. doi: 10.1002/glia.21087. PubMed PMID: 21046567.
5. Januzi L, Poirier J W, Maksoud M J E, Xiang Y Y, Veldhuizen R A W, Gill S E, Cregan S P, Zhang H, Dekaban G A, Lu W Y. Autocrine GABA signaling distinctively regulates phenotypic activation of mouse pulmonary macrophages. Cell Immunol. 2018; 332:7-23. Epub 2018 Jul. 19. doi: 10.1016/j.cellimm.2018.07.001. PubMed PMID: 30017085.
6. Tian J, Lu Y, Zhang H, Chau C H, Dang H N, Kaufman D L. Gamma-aminobutyric acid inhibits T cell autoimmunity and the development of inflammatory responses in a mouse type 1 diabetes model. J Immunol. 2004; 173(8):5298-304. PubMed PMID: 15470076.
7. Tian J, Dang H, Nguyen A V, Chen Z, Kaufman D L. Combined therapy with GABA and proinsulin/alum acts synergistically to restore long-term normoglycemia by modulating T-cell autoimmunity and promoting beta-cell replication in newly diabetic NOD mice. Diabetes. 2014; 63(9):3128-34. doi: 10.2337/db13-1385. PubMed PMID: 25146474; PMCID: PMC4141368.
8. Tian J, Dang H, O'Laco K, Song M, Tiu B—C, S G, Zakarian C, Kaufman D. Homotaurine treatment enhances CD4+ and CD8+ Treg responses and synergizes with low-dose anti-CD3 to enhance diabetes remission in type 1 diabetic mice. ImmuoHorizons. 2019: October 21; 3(10):498-510. doi: 10.4049/immunohorizons. 1900019; PMCID: PMC6823932
9. Tian J, Dang H, Wallner M, Olsen R, Kaufman D L. Homotaurine, a safe blood-brain barrier permeable GABAA-R-specific agonist, ameliorates disease in mouse models of multiple sclerosis. Sci Rep. 2018; 8 (1): 16555. Epub 2018 Nov. 10. doi: 10.1038/s41598-018-32733-3. PubMed PMID: 30410049; PMCID: PMC6224391.
10. Tian J, Dang H N, Yong J, Chui W S, Dizon M P, Yaw C K, Kaufman D L. Oral treatment with gamma-aminobutyric acid improves glucose tolerance and insulin sensitivity by inhibiting inflammation in high fat diet-fed mice. PLOS One. 2011; 6(9):e25338. doi: 10.1371/journal.pone.0025338. PubMed PMID: 21966503; PMCID: PMC3178643.
11. Vabret N, Britton G J, Gruber C, Hegde S, Kim J, Kuksin M, Levantovsky R, Malle L, Moreira A, Park M D, Pia L, Risson E, Saffern M, Salome B, Esai Selvan M, Spindler M P, Tan J, van der Heide V, Gregory J K, Alexandropoulos K, Bhardwaj N, Brown B D, Greenbaum B, Gumus Z H, Homann D, Horowitz A, Kamphorst A O, Curotto de Lafaille M A, Mehandru S, Merad M, Samstein R M, Sinai Immunology Review P. Immunology of COVID-19: Current State of the Science. Immunity. 2020. Epub 2020 Jun. 9. doi: 10.1016/j.immuni.2020.05.002. PubMed PMID: 32505227; PMCID: PMC7200337
12. Blanco-Melo D, Nilsson-Payant B E, Liu W C, Uhl S, Hoagland D, Moller R, Jordan T X, Oishi K, Panis M, Sachs D, Wang T T, Schwartz R E, Lim J K, Albrecht R A, tenOever B R. Imbalanced Host Response to SARS-CoV-2 Drives Development of COVID-19. Cell. 2020; 181(5):1036-45 e9. Epub 2020 May 18. doi: 10.1016/j.cell.2020.04.026. PubMed PMID: 32416070; PMCID: PMC7227586.
13. Hadjad J, Yatim N, Barnabei L, et a. Impaired type I interferon activity and exacerbated inflammatory responses in severe Covid-19 patients. MedRxiv. 2020; 10.1101/2020.04.19.20068015.
14. Bhandage A K, Jin Z, Korol S V, Shen Q, Pei Y, Deng Q, Espes D, Carlsson P O, Kamali-Moghaddam M, Birnir B. GABA Regulates Release of Inflammatory Cytokines From Peripheral Blood Mononuclear Cells and CD4 (+) T Cells and Is Immunosuppressive in Type 1 Diabetes. EBioMedicine. 2018; 30:283-94. Epub 2018 Apr. 9. doi: 10.1016/j.ebiom.2018.03.019. PubMed PMID: 29627388; PMCID: PMC5952354.
15. Lucas C, Wong P, Klein J, Castro T B R, Silva J, Sundaram M, Ellingson M K, Mao T, Oh J E, Israelow B, Takahashi T, Tokuyama M, Lu P, Venkataraman A, Park A, Mohanty S, Wang H, Wyllie A L, Vogels C B F, Earnest R, Lapidus S, Ott I M, Moore A J, Muenker M C, Fournier J B, Campbell M, Odio C D, Casanovas-Massana A, Yale I T, Herbst R, Shaw A C, Medzhitov R, Schulz W L, Grubaugh N D, Dela Cruz C, Farhadian S, Ko Al, Omer S B, Iwasaki A. Longitudinal analyses reveal immunological misfiring in severe COVID-19. Nature. 2020; 584 (7821): 463-9. Epub 2020 Jul. 28. doi: 10.1038/s41586-020-2588-y. PubMed PMID: 32717743.
16. De Albuquerque N, Baig E, Ma X, Zhang J, He W, Rowe A, Habal M, Liu M, Shalev I, Downey G P, Gorczynski R, Butany J, Leibowitz J, Weiss S R, McGilvray I D, Phillips M J, Fish E N, Levy G A. Murine hepatitis virus strain 1 produces a clinically relevant model of severe acute respiratory syndrome in A/J mice. J Virol. 2006; 80(21):10382-94. Epub 2006 Oct. 17. doi: 10.1128/JVI.00747-06. PubMed PMID: 17041219; PMCID: PMC1641767.
17. Khanolkar A, Hartwig S M, Haag B A, Meyerholz D K, Epping L L, Haring J S, Varga S M, Harty J T. Protective and pathologic roles of the immune response to mouse hepatitis virus type 1: implications for severe acute respiratory syndrome. J Virol. 2009; 83(18):9258-72. Epub 2009 Jul. 3. doi: 10.1128/JVI.00355-09. PubMed PMID: 19570864; PMCID: PMC2738266.
18. Khanolkar A, Hartwig S M, Haag B A, Meyerholz D K, Harty J T, Varga S M. Toll-like receptor 4 deficiency increases disease and mortality after mouse hepatitis virus type 1 infection of susceptible C3H mice. J Virol. 2009; 83(17):8946-56. Epub 2009 Jun. 26. doi: 10.1128/JVI.01857-08. PubMed PMID: 19553337; PMCID: PMC2738158.
19. Khanolkar A, Fulton R B, Epping L L, Pham N L, Tifrea D, Varga S M, Harty J T. T cell epitope specificity and pathogenesis of mouse hepatitis virus-1-induced disease in susceptible and resistant hosts. J Immunol. 2010; 185(2):1132-41. Epub 2010 Jun. 18. doi: 10.4049/jimmunol.0902749. PubMed PMID: 20554960; PMCID: PMC2897948.
20. Jin N, Kolliputi N, Gou D, Weng T, Liu L. A novel function of ionotropic gamma-aminobutyric acid receptors involving alveolar fluid homeostasis. J Biol Chem. 2006; 281(47):36012-20. Epub 2006 Sep. 28. doi: 10.1074/jbc.M606895200. PubMed PMID: 17003036.
21. Xiang Y Y, Chen X, Li J, Wang S, Faclier G, Macdonald J F, Hogg J C, Orser B A, Lu W Y. Isoflurane regulates atypical type-A gamma-aminobutyric acid receptors in alveolar type II epithelial cells. Anesthesiology. 2013; 118(5):1065-75. Epub 2013 Mar. 15. doi: 10.1097/ALN.0b013e31828e180e. PubMed PMID: 23485993.
22. Bai D, Fang L, Xia S, Ke W, Wang J, Wu X, Fang P, Xiao S. Porcine deltacoronavirus (PDCoV) modulates calcium influx to favor viral replication. Virology. 2020; 539:38-48. Epub 2019 Nov. 2. doi: 10.1016/j.virol.2019.10.011. PubMed PMID: 31670218; PMCID: PMC7112098.
23. Kraeft S K, Chen D S, Li H P, Chen L B, Lai M M. Mouse hepatitis virus infection induces an early, transient calcium influx in mouse astrocytoma cells. Exp Cell Res. 1997; 237(1):55-62. Epub 1998 Jan. 7. doi: 10.1006/excr.1997.3768. PubMed PMID: 9417866; PMCID: PMC7133765.
24. Huang T, Zhang Y, Wang C, Gao J. Propofol reduces acute lung injury by up-regulating gamma-aminobutyric acid type a receptors. Exp Mol Pathol. 2019; 110:104295. Epub 2019 Aug. 17. doi: 10.1016/j.yexmp.2019.104295. PubMed PMID: 31419406.
25. Kaynar G, Yurdakan G, Comert F, Yilmaz-Sipahi E. Effects of peripheral benzodiazepine receptor ligand Ro5-4864 in four animal models of acute lung injury. J Surg Res. 2013; 182 (2): 277-84. Epub 2012 Nov. 7. doi: 10.1016/j.jss.2012.10.023. PubMed PMID: 23127280.
26. Fortis S, Spieth P M, Lu W Y, Parotto M, Haitsma J J, Slutsky A S, Zhong N, Mazer C D, Zhang H. Effects of anesthetic regimes on inflammatory responses in a rat model of acute lung injury. Intensive Care Med. 2012; 38(9):1548-55. Epub 2012 Jun. 20. doi: 10.1007/s00134-012-2610-4. PubMed PMID: 22711173; PMCID: 20 PMC4896809.
27. Chintagari N R, Liu L. GABA receptor ameliorates ventilator-induced lung injury in rats by improving alveolar fluid clearance. Crit Care. 2012; 16(2):R55. Epub 2012 Apr. 7. doi: 10.1186/cc11298. PubMed PMID: 22480160; PMCID: PMC3681384.
28. Jin S, Merchant M L, Ritzenthaler J D, Mcleish K R, Lederer E D, Torres-Gonzalez E, Fraig M, Barati M T, Lentsch A B, Roman J, Klein J B, Rane M J. Baclofen, a GABABR agonist, ameliorates immune-complex mediated acute lung injury by modulating pro-inflammatory mediators. PLOS One. 2015; 10(4):e0121637. doi: 10.1371/journal.pone.0121637. PubMed PMID: 25848767; PMCID: PMC4388838.
29. Otomo E, Araki G, Mori A, Kurihara M. Clinical evaluation of GABA in the treatment of cerebrovascular disorders. Multi-center double-blind study in comparison with pyrithioxine and placebo. Arzneimittelforschung. 1981; 31(9):1511-23. PubMed PMID: 7030354.
30. Loeb C, Benassi E, Bo G P, Cocito L, Maffini M, Scotto P. Preliminary evaluation of the effect of GABA and phosphatidylserine in epileptic patients. Epilepsy Res. 1987; 1(3):209-12. PubMed PMID: 3143549.
31. Tower D B, Roberts E, editors. Inhibition in the Nervous System and GABA. New York: Pergamon Press; 1960.
32. Li J, Zhang Z, Liu X, Wang Y, Mao F, Mao J, Lu X, Jiang D, Wan Y, Lv J Y, Cao G, Zhang J, Zhao N, Atkinson M, Greiner D L, Prud'homme G J, Jiao Z, Li Y, Wang Q. Study of GABA in Healthy Volunteers: Pharmacokinetics and Pharmacodynamics. Front Pharmacol. 2015; 6:260. doi: 10.3389/fphar.2015.00260. PubMed PMID: 26617516; PMCID: PMC4639630.

1. A method for ameliorating a coronavirus-related, coronavirus-induced, pneumotropic virus-related, or pneumoptropic virus-induced medical condition, comprising administering to a patient in need thereof an effective amount of a GABA-receptor agonist.

2. The method of claim 1, wherein said GABA-receptor agonist is selected from the group consisting of: GABA_{A}-receptor agonists, GABA_{B}-receptor agonists, and GABA_{A-rho} receptor agonists.

3. The method of claim 2, wherein said GABA_{A}-receptor agonist is selected from the group consisting of: α₅IA, adipiplon, beta-alanine, bretazenil, CL-218,872, (−)-epigallocatechin-3-gallate, GABA, gaboxadol, homotaurine, imidazenil, isoguvacine, L-838,417, muscimol, piperidine-4-sulfonic acid, progabide, QH-ii-066, SL-651,498, taurine, zolpidem, and 3-acyl-4-quinolones.

4. The method of claim 3, wherein GABA_{B}-receptor agonist is selected from the group consisting of: baclofen, CGP-44532, GABA, gamma-hydroxybutyrate, isovaline, lesogaberan, phenibut, 3-aminopropylphosphinic acid, and 3-aminopropyl(methyl)phosphinic acid (SKF-97541).

5. The method of claim 3, wherein said GABA_{A-rho} receptor agonist is selected from the group consisting of: CACA, CAMP, and GABOB.

6. The method of claim 3, wherein an effective amount of a positive allosteric modulator (PAM) is also administered.

7. The method of claim 6, wherein said PAM is administered concurrently or in a staggered format.

8. The method of claim 6, wherein said PAM is selected from the group consisting of: alcohol (ethanol), barbiturates, benzodiazepines, BHFF, BHF-177, BSPP, certain carbamates, CGP-7930, cinacalcet, etomidate, fendiline, glutethimide, GS-39783, kavalactones, lanthanum, meprobamate, neuroactive steroids, neurosteroids, niacin/niacinamide, nonbenzodiazepines, propofol, quinazolinones, riluzole, stiripentol, theanine, thienodiazepines, valerenic acid, and volatile/inhaled anesthetics.

9. The method of claim 1, wherein said GABA-receptor agonist is GABA.

10. The method of claim 1, wherein said GABA-receptor agonist is homotaurine.

11. The method of claim 1, wherein said medical condition is related to or results from any strain of: human coronavirus, human coronavirus OC43 (“HCoV-OC43”), human coronavirus HKU1 (“HCoV-HKU1”), human coronavirus 229E (“HCoV-229E”), human coronavirus NL63 (“HCoV-NL63”), Middle East respiratory syndrome-related coronavirus (“MERS-CoV”), severe acute respiratory syndrome coronavirus (“SARS-CoV”), SARS-CoV-2, novel coronavirus, and/or other pneumotropic virus.

12. The method of claim 1, wherein said medical condition results in one or more of: death, edema, excess immune response, fever, illness, increased secretion of inflammatory factors, increased viral replication, inflammation, lethargy, pneumonia, pneumonitis, and tussis.

13. The method of claim 1, wherein said administration occurs intradermally, intramuscularly, intraperitoneally, intravenously, orally, subcutaneously, sublingually, via aerosol delivery, or via a combination of delivery routes.

14. The method of claim 13, wherein said administration occurs orally, sublingually, and/or via aerosol delivery.

15. The method of claim 1, wherein an effective amount of an anti-inflammatory compound is also administered.

16. The method of claim 15, wherein said anti-inflammatory compound is a corticosteroid.

17. The method of claim 1, wherein said coronavirus-related medical condition is COVID-19.

18. The method of claim 1, wherein said coronavirus-related medical condition is caused by infection with the SARS-CoV-2 virus.

19. A method for inhibiting coronavirus or pneumotropic virus replication in a patient, comprising administering to a patient in need thereof an effective amount of a GABA-receptor agonist;
wherein said GABA-receptor agonist is a GABA_{A}-receptor agonist, a GABA_{B}-receptor agonist, or a GABA_{A-rho} receptor agonist;
wherein said GABA_{B}-receptor agonist is selected from the group consisting of: baclofen, CGP-44532, GABA, gamma-hydroxybutyrate, isovaline, lesogaberan, phenibut, 3-aminopropylphosphinic acid, and 3-aminopropyl(methyl)phosphinic acid (SKF-97541);
wherein said GABA_{A}-receptor agonist is selected from the group consisting of: α₅IA, adipiplon, beta-alanine, bretazenil, CL-218,872, (−)-epigallocatechin-3-gallate, GABA, gaboxadol, homotaurine, imidazenil, isoguvacine, L-838,417, muscimol, piperidine-4-sulfonic acid, progabide, QH-ii-066, SL-651,498, taurine, zolpidem, and 3-acyl-4-quinolones; and
wherein said GABA_{A-rho} receptor agonist is selected from the group consisting of: CACA, CAMP, and GABOB.

20. The method of claim 19, wherein an effective amount of a positive allosteric modulator (PAM) is also administered, wherein said PAM is administered concurrently or in a staggered format, and wherein said PAM is selected from the group consisting of: alcohol (ethanol), barbiturates, benzodiazepines, BHFF, BHF-177, BSPP, certain carbamates, CGP-7930, cinacalcet, etomidate, fendiline, glutethimide, GS-39783, kavalactones, lanthanum, meprobamate, neuroactive steroids, neurosteroids, niacin/niacinamide, nonbenzodiazepines, propofol, quinazolinones, riluzole, stiripentol, theanine, thienodiazepines, valerenic acid, and volatile/inhaled anesthetics.

21. The method of claim 19, wherein said GABA-receptor agonist is GABA.

22. The method of claim 19, wherein said GABA-receptor agonist is homotaurine.

23. The method of claim 19, wherein said coronavirus or pneumotropic virus is any strain of: human coronavirus, human coronavirus OC43 (“HCoV-OC43”), human coronavirus HKU1 (“HCoV-HKU1”), human coronavirus 229E (“HCoV-229E”), human coronavirus NL63 (“HCoV-NL63”), Middle East respiratory syndrome-related coronavirus (“MERS-CoV”), severe acute respiratory syndrome coronavirus (“SARS-CoV”), SARS-CoV-2, novel coronavirus, and/or other pneumotropic virus.
